(19) United States
(12) Patent Application Publication
(10) Pub. No.: US 2019/0030318 A1
(43) Pub. Date: Jan. 31, 2019

(54) FRACTAL GEOMETRY MICROELECTRODES AND USES THEREOF

(71) Applicant: Purdue Research Foundation, West Lafayette, IN (US)

(72) Inventors: Hyowon Lee, West Lafayette, IN (US); Hyunsu Park, West Lafayette, IN (US)

(73) Assignee: Purdue Research Foundation, West Lafayette, IN (US)

(21) Appl. No.: 16/045,875

(22) Filed: Jul. 26, 2018

Related U.S. Application Data
(60) Provisional application No. 62/537,498, filed on Jul. 27, 2017.

(51) Int. Cl. A61N 1/05; G03F 7/16; B05D 1/00

(52) U.S. Cl. CPC A61N 1/05; A61N 1/0551; A61N 1/36062; G03F 7/162; B05D 1/005; A61N 1/0534

(57) ABSTRACT

A novel electrode design to prolong the lifetime and function efficacy of implantable pulse generators is disclosed herein. The novel electrode more efficiently delivers electrical charge for stimulating the nervous system, reduces power consumption by up to 50 percent while increasing functionality effectiveness. This new electrode design can be used in implantable simulation systems to treat a large number of neurological disorders with existing platforms or perform standalone.

### FIELD OF INVENTION

This disclosure is related to a novel design of electrode and the use thereof. Particularly, the Vicsek fractal electrode geometry design improves the efficiency of implantable electronic devices such as pace makers, spinal cord, peripheral nerve, or deep brain nerve stimulators.

### BACKGROUND

Electrical stimulation of the nervous system is used ubiquitously to replace and restore lost bodily functions in patients with a number of neurological impairments including neuromotor deficit, vision and hearing loss, chronic pain, and epilepsy. In 2015, the total market size for various neural stimulation devices that target spinal cord, cochlear, cerebral cortex, and other peripheral nerves (e.g., Sacral, Vagus nerve), exceeded $4.9 billion with the annual growth rate of 17%. Typically, neural stimulation devices consist of three major components: pulse generator, electrical lead, and stimulating electrodes. Since most implantable pulse generators (IPG) are powered by a rechargeable or primary cell battery inside the hermetic package, the functional lifetime of IPGs depend heavily on electrical load and device usage. Device usage is highly variable depending on patient needs, however, reducing the electrical load for stimulation can improve the performance of IPGs. The stimulation waveform output and the electrode load are primary elements of the power consumption for neurostimulation devices. IPGs provide either constant current (CC) or constant voltage (CV) stimulation waveforms to deliver electrical charge to the neural interface. The battery lifetime differs widely depending on parameters of neurostimulation (i.e., frequency, amplitude, pulse width, etc.), average IPG lifetime is approximately 4-6 years.

With advances in neurostimulation and microfabrication technologies, the demand for more precise targeting of neural substrate has fueled the development of higher density electrode arrays. For example, vision prostheses typically require more than 1000 stimulating microelectrodes with a diameter of 100 μm and manufacturers of cortical stimulation devices have begun to create higher density electrodes for stimulating various deep brain structures. The increase in electrode count leads to reduction in electrode size, which can limit the amount of charge that can be delivered through smaller electrodes. Therefore, improving resolution of stimulation outcomes using smaller microscale electrodes require careful consideration of electrode design in terms of stimulation performance as well as its impact on device longevity. A need of identifying higher efficiency and durable electrode remains.

### SUMMARY

Disclosed herein are devices with particular geometry design shapes having more efficient electrodes. These devices rely on integrated battery to deliver electrical stimulation of nervous systems. Due to the higher efficiency, the lifetime of these battery powered devices are increased. Concurrently, these highly efficient (50%) electrodes may help justify using smaller microelectrodes which can improve the electrode density for higher specificity.

This disclosure provides a microelectrode device with a fractal geometry shape. In one embodiment the fractal geometry comprising a formula of following:

Aₙ=l²·5ⁿ

Pₙ=5Pₙ₋₁−8l(P₁=12l, n=2,3,4 . . . )
wherein (Aₙ) is the area of the microelectrode, (Pₙ) is the perimeter at iteration n with l as a side of the initial square.

In some preferred embodiment the aforementioned microelectrode is configured as n=3.

In some preferred embodiment the aforementioned microelectrode is configured as l is about 8 μm.

In some preferred embodiment the aforementioned microelectrode is implantable device selected from the group consisting of pace makers, spinal cord, peripheral nerve, or deep brain nerve stimulators.

In some preferred embodiment the aforementioned microelectrode is made from platinum.

In some preferred embodiment the aforementioned microelectrode creates most current density compared to circular and serpentine type microelectrodes of similar surface area.

This disclosure further provides a method of making a fractal geometry shaped microelectrode. The method comprising the steps of:
a. Calculating a definite perimeter to area ratio pattern according to the following formula to determine the shape of the microelectrode:

Aₙ=l²·5ⁿ

Pₙ=5Pₙ₋₁−8l(P₁=12l, n=2,3,4 . . . );
b. Spun coating a photoresist layer over a silicon nitride layer with the defined pattern in step a;
c. Depositing a Platinum film about 100 nm thick on to the photoresist layer using a titanium (about l0nm) as an adhesion layer;
d. Lifting off the photoresist layer to create the electrode arrays; and
e. Spun coating about 1.5 μm thick of polyimide over the electrode array as a passivation layer to cure.

In some preferred embodiment the aforementioned method uses silicon nitride layer of about 500nm.

In some preferred embodiment the aforementioned method further comprising applying an adhesion promoter before spun coating polyimide.

These and other features of the invention will become more apparent with the drawings, detailed description and claims.

### BRIEF DESCRIPTION OF DRAWINGS

FIG. 1. Microelectrode geometry (A) Planar geometry with different shapes. (B) Design of the Vicsek fractal at different iteration.

FIG. 2. Fabrication sequence of microelectrode arrays (a) Deposition of 500 nm silicon nitride (b) Evaporation of Ti/Pt (c) Polyimide passivation layer coating followed by RIE for opening.

FIG. 3. Geometry of the finite element model for average current density distribution on the hemisphere boundary region on the microelectrodes when a constant voltage of −0.6 V was applied. (COMSOL Multiphysics)

FIG. 4. Experimental setup for the measurements. (A) A 3D schematic diagram of the MEA packaging platform for electrochemical measurements. (B) An assembled measurement setup with the reference electrode.

FIG. 5. Representative voltage transient of microeletrode with biphasic, symmetrical current pulse was applied at 50 Hz highlighted with maximum negative potential excursion (E_{mc}) and maximum driving voltage (V_{dr}).

FIG. 6. Optical micrographs of the fabricated microelectrodes (scale bar=50 μm)

FIG. 7. FEM results of average current density(A) and total delivered current on hemi-sphere boundaries(B).

FIG. 8. Comparison of cyclic voltammogram of Pt microelectrodes with different shape in PBS.

FIG. 9. Charge storage capacity of the microelectrodes. ANOVA results revealed significant differences (p<0.01) as compared to electrode #1 (*), and significant differences (p<0.01) between fractal and serpentine I, serpentine I and serpentine II.

FIG. 10. Impedance spectra of microelectrodes with different shapes. The measurement was made over a frequency range from 10 Hz to 100 kHz with sinusoidal voltage excitation with 10 mV.

FIG. 11. Comparison of the voltage-transient response of different shaped microelectrodes in a pulse with constant charge per phase.

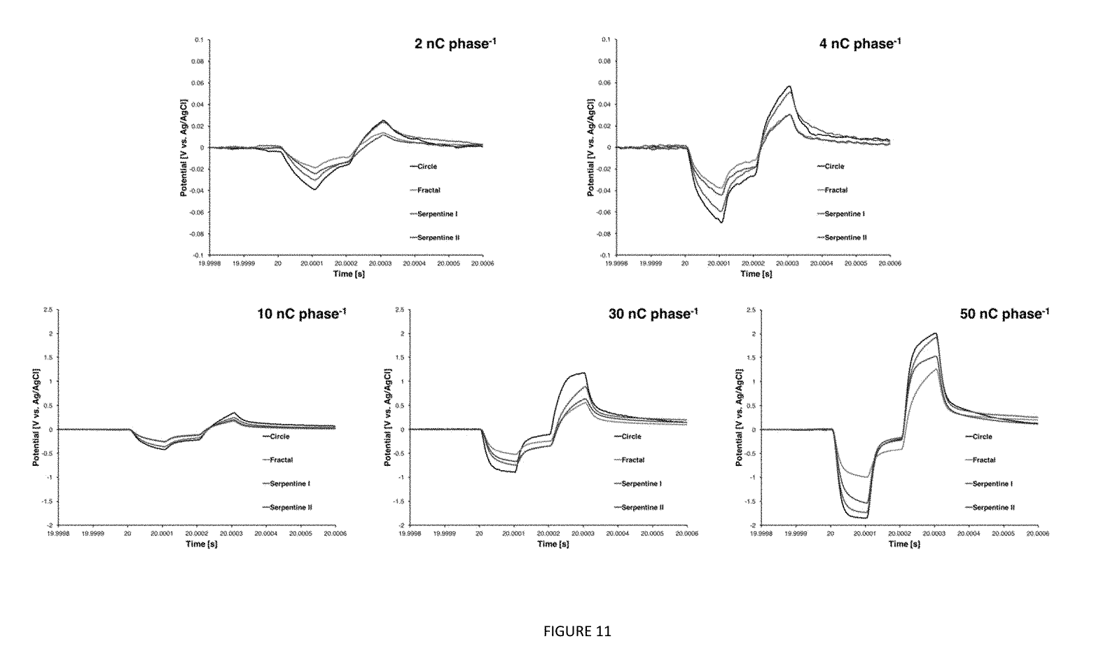

FIG. 12. Maximum negative potential excursion of the microelectrodes with different shape. Post-hoc pairwise comparisons using Tukey's test (p<0.01)

FIG. 13. Maximum driving voltage. Post-hoc pairwise comparison using Tukey's test (p<0.01)

FIG. 14. Comparison of load energy of the microelectrodes. (A) Energy consumption for a single cathodal pulse from the microelectrodes with different shape when the constant charge per phase was injected (B) Energy consumption compared to the circular shaped microelectrode.

### DETAILED DESCRIPTIONS

There have been various attempts to increase the stimulation efficiency of the neurostimulator by decreasing microelectrode impedance or increasing the charge transfer capacity. For instance, the surface morphology of the microelectrode and material was found to affect electrochemical impedance and charge transfer capacity. Subramaniam et al., reported that microelectrode coated with poly(3,4-ethylenedioxythiophene) (PEDOT) has higher charge injection limits than iridium oxide (IrOₓ) and platinum iridium microelectrode in vitro and in vivo (Venkatraman et al. 2011). Sandeep et al., investigated that sputtered iridium oxide is superior than activated iridium oxide because of the difference in morphology of the iridium oxide surface (Negi et al. 2012). C. de Haro et al., studied that electroplated platinum has lower impedance and higher corrosion resistance than sputtered platinum, which can improve the lifetime of the microelectrode (De Haro et al. 2002). Shota et al., showed that microelectrode composed of IrOₓ/platinum (Pt)-black with nanoscale roughness has a lower impedance and high charge-injection capability than flat microelectrode (Yamagiwa et al. 2015). C. Boehler et al., reported that Pt microelectrode with nanograss structure has reduced impedance and strong adhesion to metallized substrate (Boehler et al. 2015). However, diffusion limitations is thought to prevent ionic charge transfer to the full surface area during typical stimulation frequency which dampened practical improvement in vivo (He et al. 2009).

The dependence of the impedance and charge injection capacity on microelectrode size and geometry has previously been reported. Electrochemical impedance spectroscopy (EIS) from different sized circular shaped microelectrode showed that the smaller microelectrode had higher impedance with increase in the solution resistance and shorter charging time of capacitive double-layer on electrode (Ahuja et al. 2008). M. Grill et al., reported that electrodes split to segments with higher perimeter-to-area ratio have demonstrated higher stimulation efficiency with reduced power requirements of IPGs, although there is no significant difference in impedance between single electrode and segmented one (Grill & Wei 2009; Wei & Grill 2009; Howell & Grill 2014; Butson et al. n.d.). Cogan et al. demonstrated that increasing the perimeter-to-area ratio of the microelectrode lowered the electrode impedance and improved the charge injection limit due to decrease of the faradaic resistance and higher ion flux to the electrode surface (Cogan et al. 2014). However, they reported only minimal improvement in electrode performance due to change in perimeter-to-area ratio. The effect of perimeter-to-area ratio on electrochemical properties of electrode warrants additional investigation because, even though the shape with increased perimeter in microelectrodes can lead to significant non-uniformity in current density, the edges near center of the electrode still have significantly smaller current density than outer edges (Online et al. 2017). Furthermore, Atefeh et al., showed that the base boundaries of star shape and inner boundaries of the spiral electrode do not contribute to production of maximum current density even they have high perimeter-to-area ratio (Ghazavi et al. 2015).

Here we examined the role of electrode geometry in terms of perimeter-to-area (PSA) ratio and shape using custom microfabricated electrode arrays. Four types of electrodes were created: circular, fractal, serpentine I, and serpentine II. The surface area and perimeter of typical circle microelectrode with 100 μm diameter was selected as a standard when the other shapes were designed. The fractal shape is useful when a shape with long perimeter but defined area is needed. In terms of electrode geometry design, fractal shape can increase the stimulation efficiency from the increase of the spatial derivative of the current density (Wei et al. 2015; Wei et al. 2016). Serpentine design is the one of shapes having high PSA ratio and has potential to use it for flexible substrate based on its elastic mechanics (Xu et al. 2013; Yang et al. 2015; Fan et al. 2014). Delivered current and current density around the different shaped microelectrode were quantified by using finite element model simulation. Each electrode was characterized using cyclic voltammetry, EIS. The cathodal and the total charge storage capacity of each electrode were calculated from the time integral of the current in cyclic voltammogram, which is related to the charge injection capability of electrode. To investigate the effects of the geometry on charge injection limit, we compared the maximum negative potential excursion and the maximum driving voltage of the different shaped microelectrodes through the voltage transient with different charge injection. Finally, energy consumption from different shaped microelectrodes was quantified by utilizing cathodic potential transient and applied current pulse. Our results indicate that the electrode shape may play a significant role in charge injection capability of microelectrodes. We have found that electrodes with same PSA and SA resulted in significantly different electrode performance which may facilitate optimization in designing a more energy efficient stimulating electrodes.

### Methods

### Electrode Design

A description of the platinum (Pt) electrode geometries is provided in Table 1.

TABLE 1
Dimensions of the microelectrodes.

| | | Circle | Fractal | Serpentine I | Serpentine II |
|---|---|---|---|---|---|
| As Designed | Perimeter [mm] | 0.3142 | 1.998 | 1.998 | 3.156 |
| | Area [mm²] | 7.854 × 10⁻³ | 7.854 × 10⁻³ | 7.854 × 10⁻³ | 7.854 × 10⁻³ |
| | Perimeter/Area [mm⁻¹] | 40 | 254 | 254 | 400 |
| Measured | Perimeter [mm] | 0.3016 | 1.886 | 1.912 | 3.078 |
| | Area [mm²] | 7.238 × 10⁻³ | 7.214 × 10⁻³ | 7.357 × 10⁻³ | 7.573 × 10⁻³ |
| | Perimeter/Area [mm⁻¹] | 42 | 261 | 259 | 406 |
| | Outer Perimeter/ | 42 | 230 | 150 | 205 |
| | Area [mm⁻¹] | | | | |

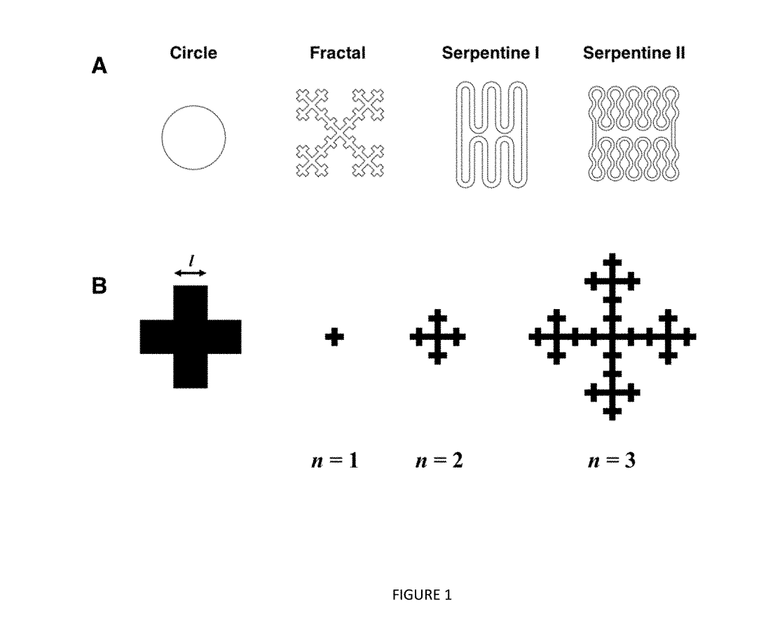

The geometries of electrode are designed based on the surface area from circular shaped microelectrode with diameter of 100 μm (7854 μm²) (FIG. 1A). The fractal shape was designed based on the Vicsek fractal which can make zero area with infinite perimeter. In the Vicsek fractal, the area (Aₙ) and perimeter (Pₙₛ) at iteration n with l as a side of initial square follow the equations below.

Aₙ=l²·5ⁿ

Pₙ=5Pₙ₋₁−8l(P₁=12l, n=2,3,4 . . . )

Based on the area of circle microelectrode (7853 μm²) and minimum feature size which is proper for microfabrication and pattern alignment, the side of the smallest square unit of the fractal is designed as 7.93 μm with three iteration. The specific perimeter-to-area ratios in serpentine I and II were achieved by adjusting the radius of the grooved parts and length of the connection parts with straight line.

Fractal shape and serpentine I shape have approximately 6.35 times longer perimeter than circular shape, but have same surface area. Serpentine II shape has highest perimeter-to-area ratio with 10 times larger perimeter than circular one.

### Electrode Fabrication

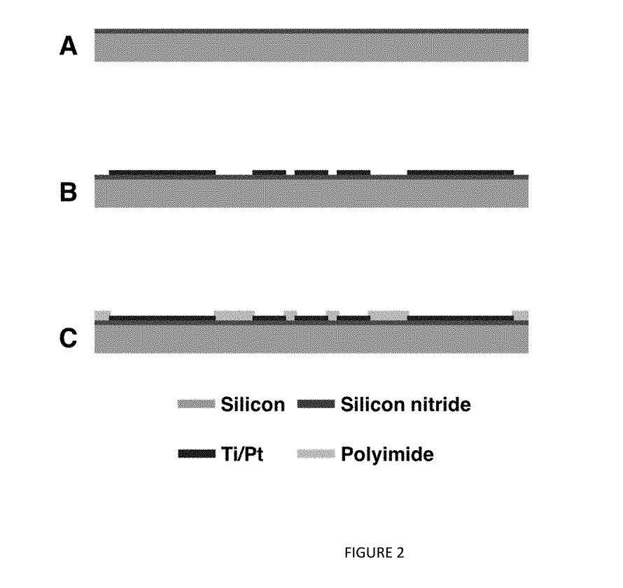

FIG. 2 illustrates the overall fabrication flow. Platinum microelectrode of varying perimeter-to-area ratio were fabricated on 500 nm film of silicon nitride layer by plasma enhanced chemical vapor deposition (Axic, Milpitas, Calif., USA). A photoresist (AZ1518, MicroChem, Newton, Mass., USA) was spun coated over the silicon nitride layer and patterned to define microelectrode designs with different shapes. Platinum film (100 nm thick) was deposited on to the photoresist using a titanium (10 nm) as an adhesion layer. The electrode arrays were created by lift off the photoresist. A 1.5 μm thick of polyimide (PI-2545, HD Microsystems, Parlin, N.J.) was spun-coated over the wafer and cured as a passivation layer. Before the application of the polyimide, adhesion promoter (VM-652, HD Microsystems, Parlin, N.J.) was used. The microelectrodes, counter electrode, and contact pads were created by reactive ion etching (RIE) with 20 sccm O₂ at 150 W in 100 mTorr for 10 min using photoresist (AZ9260, MicroChem, Newton, Mass., USA) as a mask.

### Finite Element Model (FEM) for Simulation of Current Density Distribution

The FEM model was implemented using the electric current mode of COMSOL 5.2a (COMSOL Inc., USA). The electric current mode of COMSOL software solved the charge conservation equation for calculating current density distribution across the internal boundaries below.

$$∇ \cdot J + \frac{\partial ρ}{\partial t} = 0$$

where J is the current density, and ρ is the charge density. The current density is governed by the equations below:

$$J = σ E = - σ ∇ V$$

$$∇ \cdot J + \frac{\partial ρ}{\partial t} = {∇}^{2} V = 0$$

with the electrical potential, V.

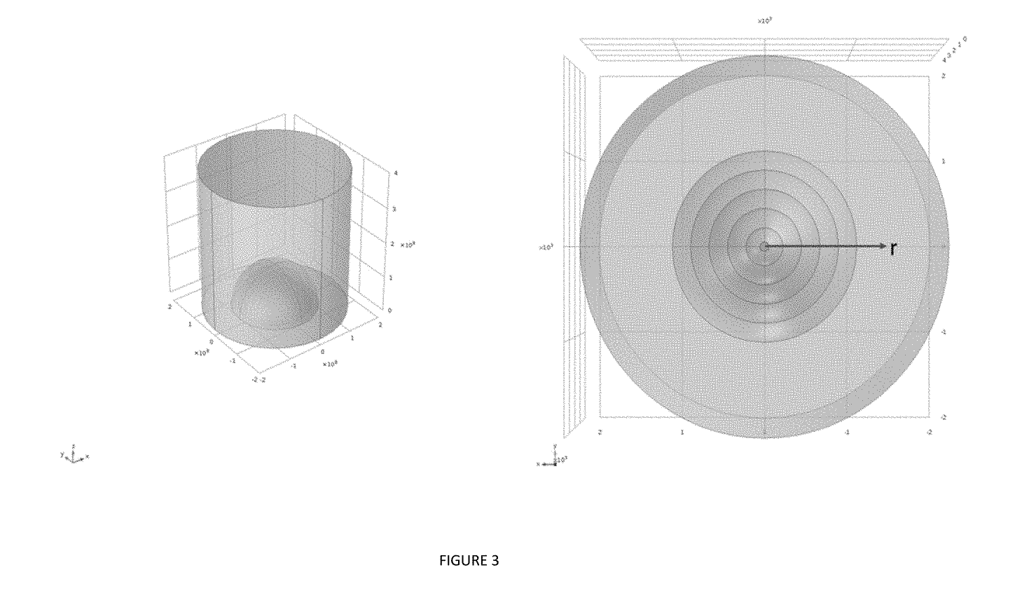

The electric currents mode assumed that there is no Faradaic current through chemical reactions on the electrode surface. The model includes microelectrode domain, extracellular boundary with cylindrical shape, and five hemi-sphere domains with radius from 200 μm to 1200 μm to estimate current density distribution and total delivered current around the electrode (FIG. 3). The conductivity of simulated domain was 0.2 S m⁻¹ which is matched with the brain tissue conductivity. Applied potential on the electrode surface is −0.6 V which is negative potential limit in water window. The surrounding cylindrical boundary was grounded with 0 V, and the model was meshed using tetrahedral mesh elements.

### Cyclic Voltammetry and Electrochemical Spectroscopy

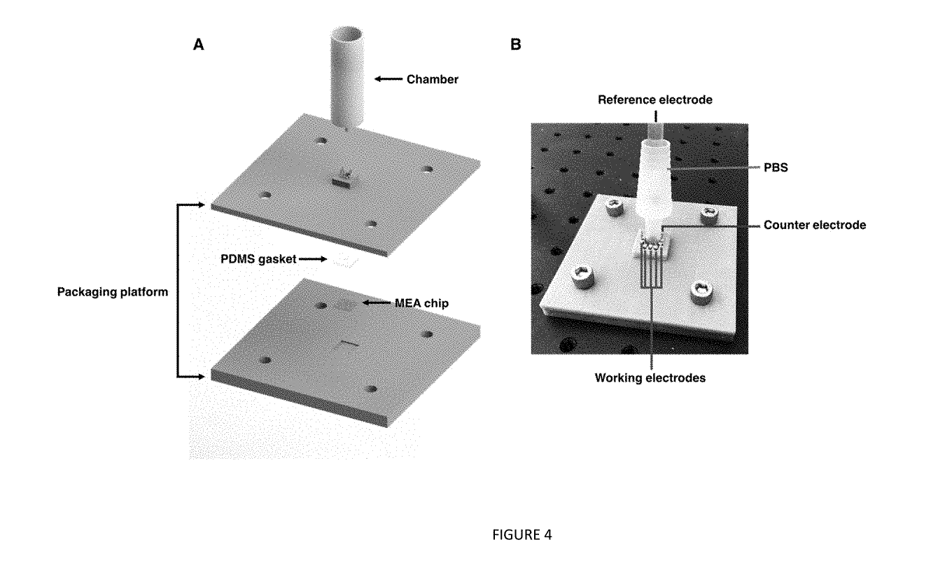

Cyclic voltammetry (CV) and electrochemical spectroscopy was performed using the customized microelectrode packaging platform (FIG. 4). CV was measured by a Potentiostat (SP-200, BioLogic.Inc, Seyssinet-Pariset, France) in a standard three-electrode configuration using KC1 saturated Ag|AgCl (RE-1CP, ALS Co., Ltd, Tokyo, Japan), and working and counter electrode on the device. CV was performed in phosphate-buffered saline solution (PBS) having composition of KH₂PO₄ 1.1 mM, NaCl 155 mM, and Na₂HPO₄.7H₂O 3 mM with pH 7.4 (ThermoFisher Scientific, Waltham, Mass., USA). All CVs were measured at 50 mV s⁻¹ sweep rate between potential range of −0.65 V and 0.85 V versus Ag|AgCl reference electrode which is confined by the water electrolysis window of platinum electrode. The EIS measurements were performed using the same potentiostat. The pertubation potential was sinusoidal 10 mV excitation voltage with the frequency range from 10 to 10⁵ Hz.

### Voltage Transient Measurements

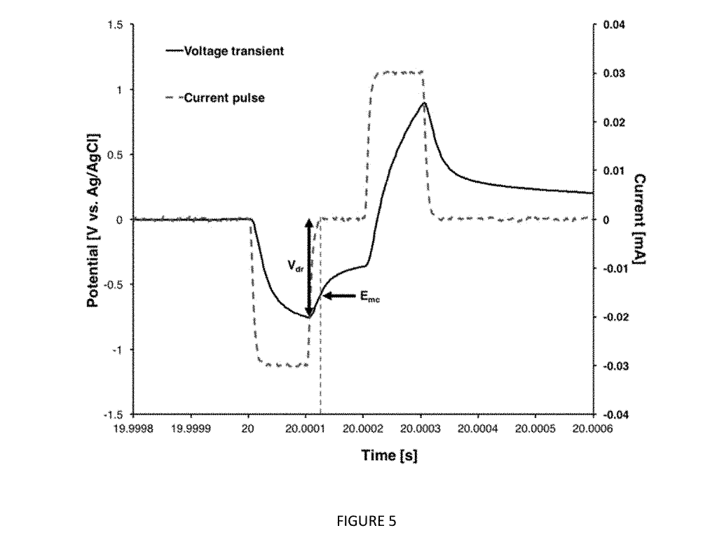

The biphasic current pulsing for voltage transients were performed with an analog stimulus isolator (AM 2200, AM Systems, Sequim, Wash., USA). We used a customized MATLAB program (R2016a, Mathworks, Natick, Mass., USA) to design injected pulse with having specific pulse width, amplitude, and frequency for experiment. The designed pulses were injected into the electrode-electrolyte test cell, and a data acquisition board (NI USB-6353, National Instruments, Austin, Tex., USA) was used to interface with the program to record the voltage transient response. The biphasic pulse used in the experiments were cathodic-first current pulse with 100 μs duration followed by 100 μs inter-phase delay at 50 Hz. The maximum negative potential excursion (E_{mc}) is the potential immediately after the end of the cathodic pulse (FIG. 5). The measured time delay that the current becomes zero was about 12 μs, and E_{mc} was taken as the potential at 12 μs after the end of the cathodic current pulse.

### Results

### Fabrication Results

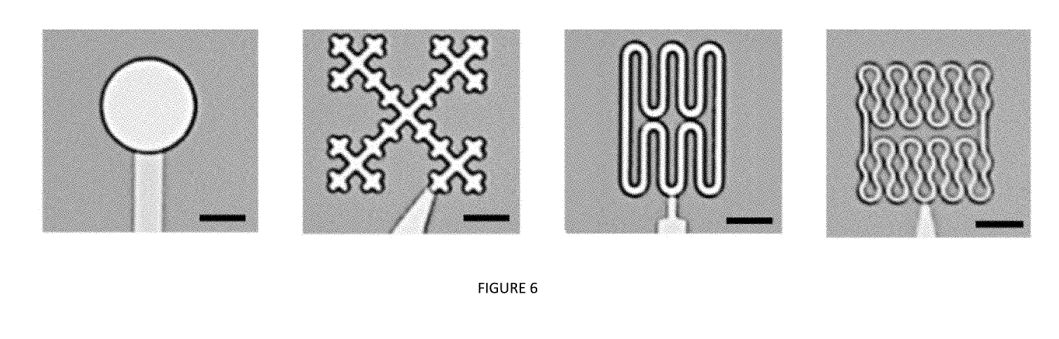

The geometry of fabricated microelectrodes is shown in the optical microscope in FIG. 6. Difference of perimeter and area between design and actually measured value results from under-etched area in microfabrication (Table 1).

### Current Density Distributions With Three-Dimensional Finite Element Model

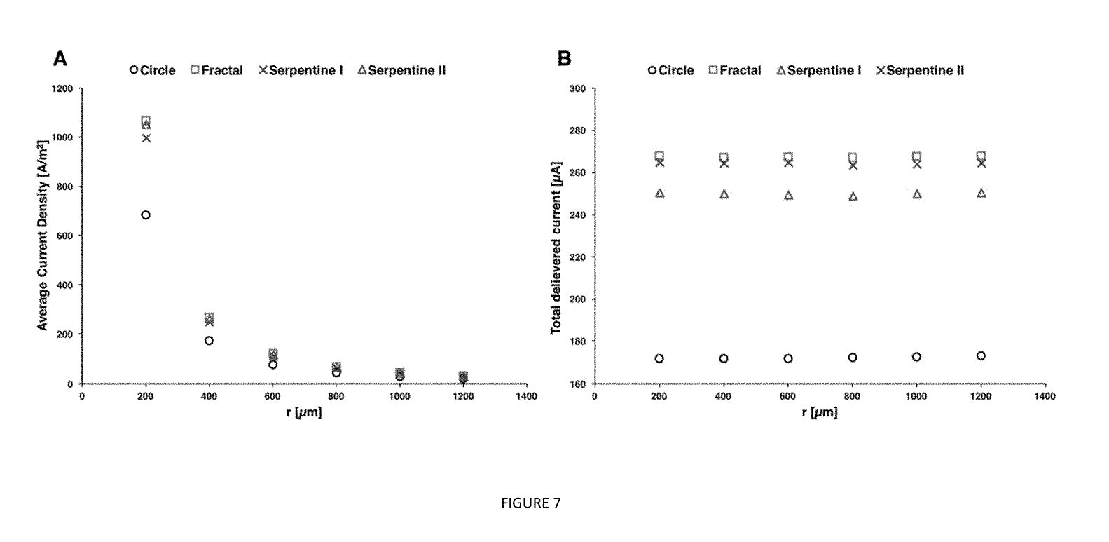

The current density distribution and total delivered current on the hemi-sphere shaped boundary with applied potential of −0.6 V was studied for different shaped microelectrodes. FIG. 7A shows that fractal produces the highest current densities around the microelectrode, and the current density of the other shapes follows in the order of perimeter-to-area ratio. As illustrated in FIG. 7B, the fractal shape delivered largest total current of 267 μA. The serpentine II and serpentine I delivered the current of 264 μA and 250 μA, respectively. The minimum current was delivered through the circular microelectrode with 172 μA.

Cyclic voltammetry

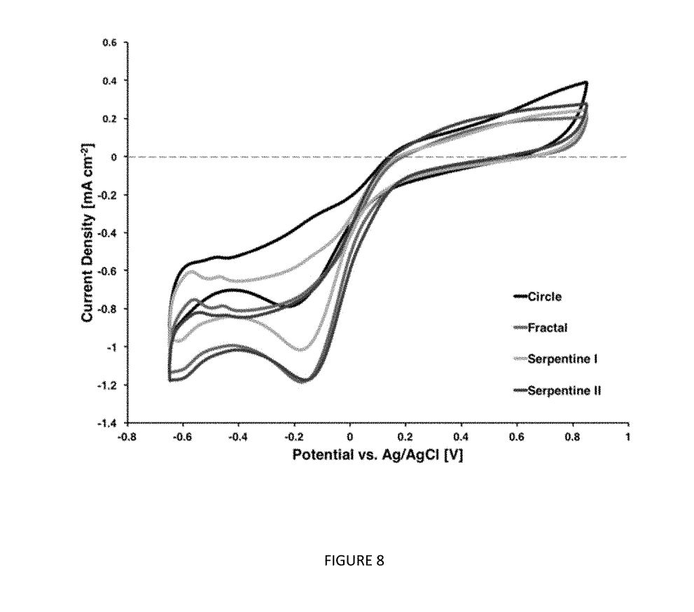

FIG. 8 shows CV response of the microelectrodes with different shapes (Circle, Fractal, Serpentine I, and Serpentine II) measured in PBS from −0.65 V to 0.85 V at a sweep rate of 50 mV s⁻¹. It can be seen in the CV that the fractal, serpentine I, and serpentine II shaped microelectrodes with higher perimeter-to-area ratio have lower cathodic current density than circular shaped microelectrode when the microelectrodes potential decreased in negative direction below 0 V. The microelectrodes were characterized by calculating their cathodal charge storage capacity and total charge storage capcity (CSC_{c} and CSCₜ). The charge storage capacity was calculated by following equation (Cogan 2008):

$$CSC = \frac{1}{vA} {\int }_{{E}_{c}}^{{E}_{a}} \vert i \vert dE ( C \text{/} {cm}^{2} )$$

where, E is the potential versus Ag|AgCl reference electrode, i is the measured current, Ea and Ec are the positive and negative potential range, respectively, A is the surface area of the microelectrode and v is the scan rate. For CSC_{c}, the cathodic current was only used for calculation,

TABLE 2
CSC_{c} and CSCₜ of microelectrode

| | Circle | Fractal | Serpentine I | Serpentine II |
|---|---|---|---|---|
| CSC_{c} | 3.69 ± 0.31 | 5.79 ± 0.1 | 4.62 ± 0.1 | 6.13 ± 0.55 |
| [mC cm⁻²] | | | | |
| CSCₜ | 5.42 ± 0.39 | 8.51 ± 0.13 | 6.37 ± 0.13 | 8.94 ± 0.79 |
| [mC cm⁻²] | | | | |

and the both of anodic and cathodic current were used for calculating CSCₜ.

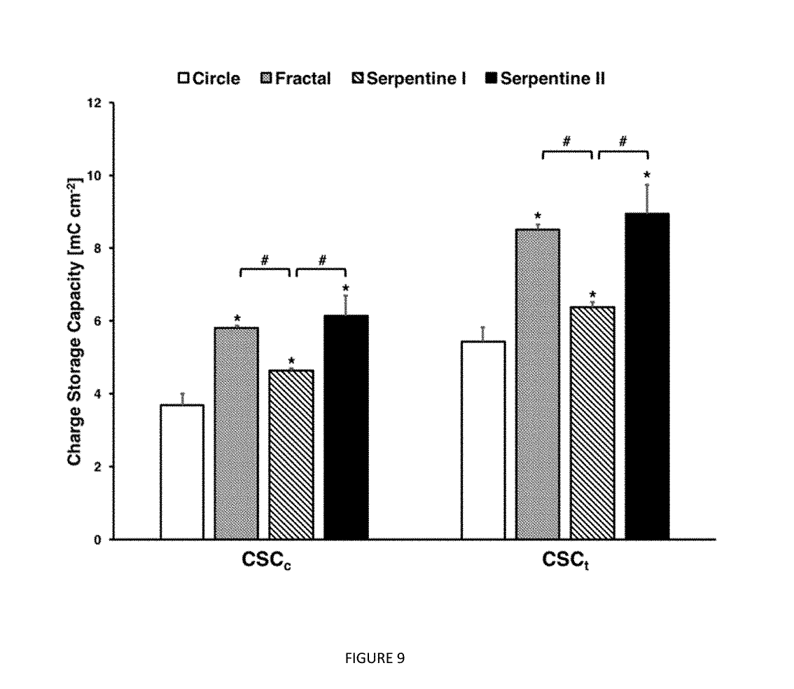

The mean cathodal charge storage capacity (CSC_{c}) and total charge storage capacity (CSCₜ) calculated from the integral of the cathodic current in a CV response over a negative potential range and from −0.65 V to 0.85 V, respectively. The average CSC and estimated mean CSC was shown in Table 2. The CSC and CSC of the electrodes were compared using one-way ANOVA with Tukey's HSD post-hoc analyses. The results showed that the CSC_{c} (and CSCₜ) of circular microelectrode significantly smaller than the other microelectrodes with higher perimeter-to-area ratio (p<0.01) (FIG. 9). However, there were significant difference between CSC_{c} (and CSCₜ) of the fractal shaped microelectrode and the serpentine I shaped microelectrode having same perimeter-to-area ratio. Furthermore, fractal microelectrode and serpentine II shaped one did not have a statistically significant difference in CSC_{c} (and CSCₜ) although the serpentine II shaped microelectrode has 1.6 times higher perimeter-to-area ratio.

### Impedance of the Microelectrodes

The impedance measured at high frequency is dominated by the series resistance between working and reference electrode which is the resistance made by current moving out into solution, and the electrode-electrolyte interface impedance contributes to the impedance at low frequency (Ragheb et al. 1992). We compared the impedance at 10 Hz, 1 kHz, and 100 kHz to study the effect of the electrode geometry on impedance using one-way ANOVA (=0.01). However, there was no significant difference between the impedances of circle, fractal, serpentine I, and serpentine II at every frequency.

### Voltage Transient Measurement

The voltage transient response from the different shaped microelectrodes was compared in three different constant injected charge (2 nC, 4 nC, 10 nC, 30 nC, and 50 nC) per phase at a frequency of 50 Hz (FIG. 9). The biased potential in the interphase region was 0 V versus Ag|AgCl. The charge level with 30 nC/phase was not enough to polarize the fractal electrode to −0.6 V limit in water window, but serpentine I , II and circular shaped were over-polarized in 30 nC/phase based on the full potential range where water reduction might occur.

### Maximum Negative Potential Excursion

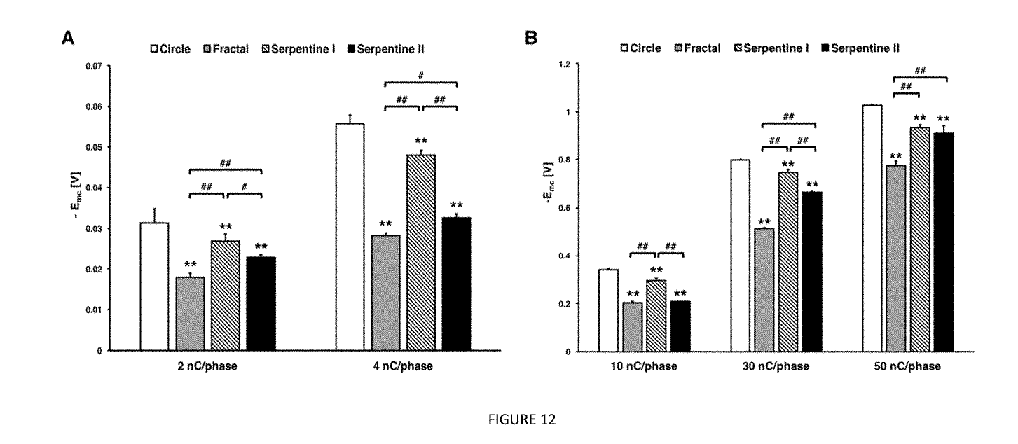

A comparison of the E_{mc} is shown in FIG. 12. Post-hoc pairwise comparison using Tukey' s test indicated that the microelectrodes with fractal and serpentine II shapes had significantly higher E_{mc} than circular electrode. However, there was no significance difference between the E_{mc} of serpentine I and circular microelectrode with 10 nC/phase charge injection. Furthermore, serpentine I shaped microelectrode had significantly lower E_{mc} than fractal and serpentine II shaped microelectrodes when 2 nC, 4 nC, and 10 nC charge was injected for a cathodic phase. When charge injected with 30 nC and 50 nC per phase, serpentine I shaped electrode not only had also significantly higher E_{mc} than circular shaped one, but significantly lower E_{mc} than fractal and serpentine II shaped one even though they had same and higher perimeter-to-area ratio. Furthermore, fractal one always had significantly highest E_{mc} than the other shaped electrodes.

### Maximum Driving Voltage

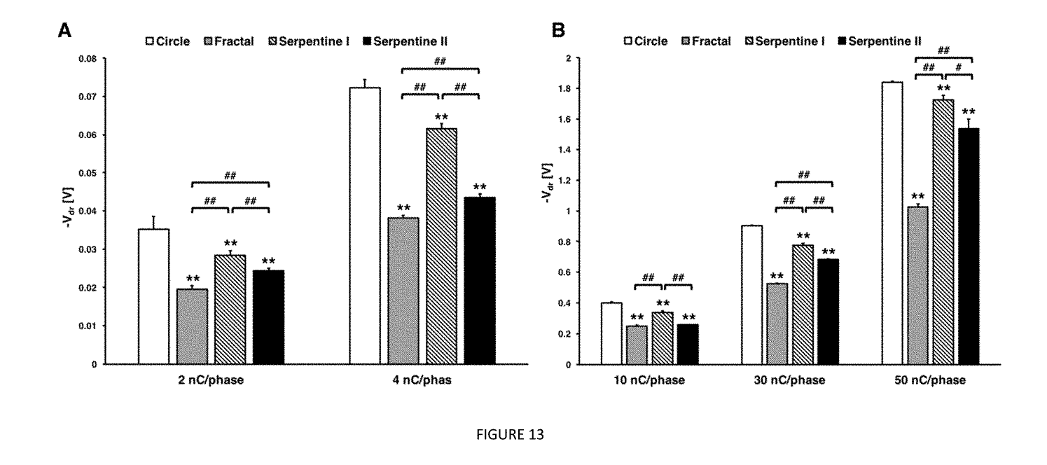

The maximum driving voltage (V_{dr}) is the maximum voltage required to deliver the current pulse, which is related to the energy required to deliver the pulse. The V_{dr} required to inject constant charge per phase from the different shaped electrode is shown in FIG. 13. One-way ANOVA with post-hoc pairwise comparison using Tukey's test showed that the circular electrode needs lowest maximum voltage for applying 2 nC, 4 nC, 10 nC, 30 nC, and 50 nC per phase compared to the other electrodes. However, there was no significant difference between V_{dr} of serpentine I and circular electrode with 10 nC per phase injection. Fractal electrode needs less maximum voltage than the other shaped electrodes. Even serpentine II shape has highest perimeter-to-area ratio, V_{dr} of the serpentine II was significantly smaller than fractal one, but still higher than serpentine I microelectrode.

### Energy Consumption

The energy required to apply a cathodal pulse is described by the equation below (Foutz et al. 2012):

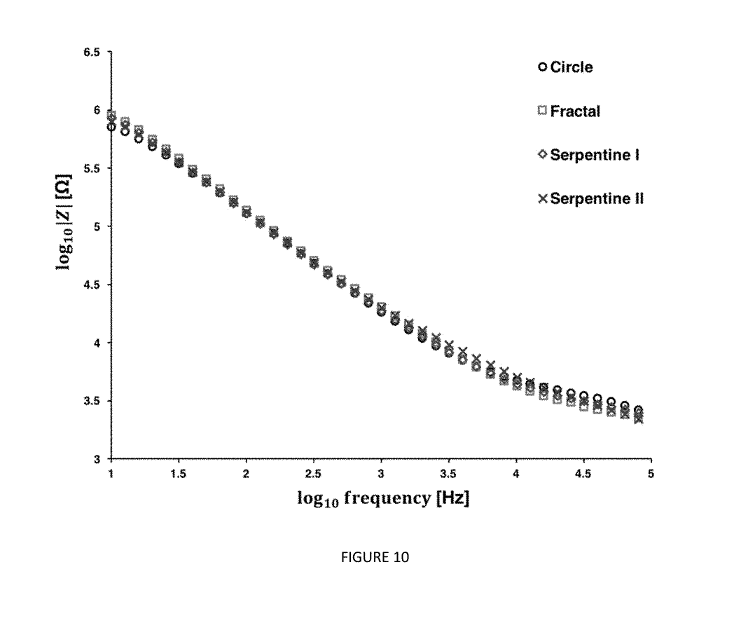

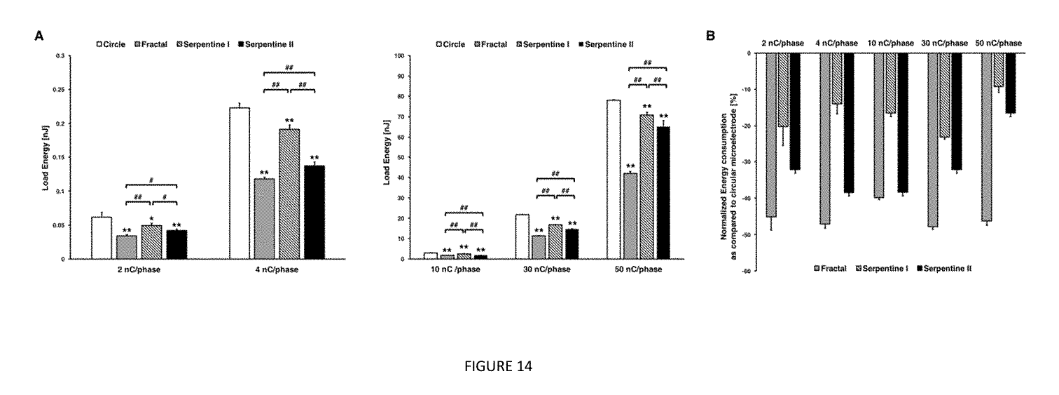

E_{load}=∫₀^{PW}IₛₜᵢₘV_{load}dt

where E_{load} is the energy consumed in the electrode and the solution, Iₛₜᵢₘ is the current amplitude for the pulse, V_{load} is the load voltage, and PW is the pulse-width. A comparison of the required energy for applying same amount of the current pulse is shown in FIG. 10. The circular shaped electrode needed significantly higher energy for applying same amount of the current pulse than the other shaped microelectrodes. When 10 nC was injected for each different shaped microelectrode, there was no significance difference between dissipated energy of circle and serpentine I shaped microelectrode. Among the microelectrode with higher perimeter-to-area ratio than circle shaped one, the fractal shaped one had significantly lowest energy requirement for forming same amount of the current pulse injection than the other shaped electrodes although the perimeter-to-area ratio is smaller than serpentine II and same with serpentine I (FIG. 14).

### REFERENCES

Ahuja, A. K. et al., 2008. The dependence of spectral impedance on disc microelectrode radius. IEEE Transactions on Biomedical Engineering, 55(4), pp. 1457-1460.
Boehler, C., Stieglitz, T. & Asplund, M., 2015. Nanostructured platinum grass enables superior impedance reduction for neural microelectrodes. Biomaterials, 67, pp. 346-353. Available at: http://dx.doi.org/10.1016/j.biomaterials.2015.07.036.
Butson, C. R. et al., Role of electrode design on the volume of tissue activated during deep brain stimulation.
Cameron, T., 2004. Safety and efficacy of spinal cord stimulation for the treatment of chronic pain: a 20-year literature review. J Neurosurg, 100(3 Suppl Spine), pp. 254-267. Available at: http://www.ncbi.nlm.nih.gov/pubmed/15029914.
Cogan, S. F., 2008. Neural stimulation and recording electrodes. Annual review of biomedical engineering, 10, pp. 275-309. Available at: http://www.ncbi.nlm.nih.gov/pubmed/18429704.
Cogan, S. F., Ehrlich, J. & Plante, T. D., 2014. The effect of electrode geometry on electrochemical properties measured in saline. Conference proceedings: . . . Annual International Conference of the IEEE Engineering in Medicine and Biology Society. IEEE Engineering in Medicine and Biology Society. Annual Conference, 2014, pp. 6850-6853.
Fan, J. a et al., 2014. Fractal design concepts for stretchable electronics. Nature communications, 5, p. 3266. Available at: http://www.ncbi.nlm.nih.gov/pubmed/24509865.
Fisher, R. et al., 2010. Electrical stimulation of the anterior nucleus of thalamus for treatment of refractory epilepsy. Epilepsia, 51(5), pp. 899-908.
Foutz, T. J. et al., 2012. Energy Efficient Neural Stimulation: Coupling Circuit Design and Membrane Biophysics. PLoS ONE, 7(12).
Ghazavi, A. et al., 2015. Effect of planar microelectrode geometry on neuron stimulation: Finite element modeling and experimental validation of the efficient electrode shape. Journal of Neuroscience Methods, 248, pp. 51-58. Available at: http://dx.doi.org/10.1016/j.jneumeth.2015.03.024.
Grill, W. M. & Wei, X. F., 2009. High efficiency electrodes for deep brain stimulation. Engineering in Medicine and Biology Society, 2009. EMBC 2009. Annual International Conference of the IEEE, pp. 3298-3301.
De Haro, C. et al., 2002. Electrochemical platinum coatings for improving performance of implantable microelectrode arrays. Biomaterials, 23(23), pp. 4515-4521.
He, C. et al., 2009. Electrochemical Impedance Spectroscopy Characterization of Electron Transport and Recombination in ZnO Nanorod Dye-Sensitized Solar Cells, pp. 10322-10325.
Howell, B. & Grill, W. M., 2014. Evaluation of high-perimeter electrode designs for deep brain stimulation. Journal of Neural Engineering, 11(4), p. 46026. Available at: http://stacks.iop.org/1741-2552/11/i=4/a=046026?key=crossref.d43dd048172cf6ff40d5f9da53afb0c3.
Jimbo, Y. et al., 2003. A System for MEA-Based Multisite Stimulation., 50(2), pp. 241-248.
Jimbo, Y., 1992. Electrical stimulation and recording from cultured neurons using a planar electrode array., 29, pp. 193-204.
Kral, A. & Sharma, A., 2012. Developmental neuroplasticity after cochlear implantation. Trends in Neurosciences, 35(2), pp. 111-122. Available at: http://dx.doi.org/10.1016/j.tins.2011.09.004.
Li, X. et al., 2015. The injectable neurostimulator: an emerging therapeutic device. Trends in Biotechnology, 33(7), pp. 388-394. Available at: http://dx.doi.org/10.1016/j.tibtech.2015.04.001.
Luan, L. et al., 2017. Ultraflexible nanoelectronic probes form reliable, glial scar—free neural integration. Science Advances, 3(e1601966), pp. 1-10.
Negi, S., Bhandari, R. & Solzbacher, F., 2012. Morphology and Electrochemical Properties of Activated and Sputtered Iridium Oxide Films for Functional Electrostimulation*. Journal of Sensor Technology, 2(September), pp. 138-147. Available at: http://dx.doi.org/10.4236/jst.2012.23020%5Cnhttp://www.SciRP.org/journal/jst.
Online, B. E. et al., 2017. Estimation of current density distribution under electrodes for external defibrillation, (June).
Patil, A. C. & Thakor, N. V., 2016. Implantable neurotechnologies: a review of micro- and nanoelectrodes for neural recording. Medical and Biological Engineering and Computing, 54(1), pp. 23-44.
Pikov, V., 2015. Global market for implanted neuroprostheses, Elsevier Ltd. Available at: http://www.sciencedirect.com/science/article/pii/B9781782421016000185.
Ragheb, T. et al., 1992. The impedance of a spherical monopolar electrode. Annals of Biomedical Engineering, 20(6), pp. 617-627.
Riesen, C. Van et al., 2016. Disease-specific longevity of impulse generators in deep brain stimulation and review of the literature. Journal of Neural Transmission, 123(6), pp. 621-630.
Rossi, P. J. et al., 2016. Proceedings of the Third Annual Deep Brain Stimulation Think Tank: A Review of Emerging Issues and Technologies. Frontiers in Neuroscience, 10(April), p. 119.
Shendkar, C. V. et al., 2015. Therapeutic effects of functional electrical stimulation on gait, motor recovery, and motor cortex in stroke survivors. Hong Kong Physiotherapy Journal, 33(1), pp. 10-20. Available at: http://dx.doi.org/10.1016/j.hkpj.2014.10.003.
Venkatraman, S. et al., 2011. In Vitro and In Vivo Evaluation of PEDOT Microelectrodes for Neural Stimulation and Recording, 19(3), pp. 307-316.
Ventura, R. E., Balcer, L. J. & Galetta, S. L., 2014. The neuro-ophthalmology of head trauma. The Lancet Neurology, 13(10), pp. 1006-1016. Available at: http://dx.doi.org/10.1016/S1474-4422(14)70111-5.
Wei, X. et al., 2016. Novel Fractal Planar Electrode Design for Efficient Neural Stimulation, pp. 1802-1805.
Wei, X. F. & Grill, W. M., 2009. Analysis of high-perimeter planar electrodes for efficient neural stimulation. Frontiers in neuroengineering, 2(November), p. 15.
Wei, X. F., Iyengar, N. & Demaria, A. H., 2015. Iterative Electrodes Increase Neural Recruitment for Deep Brain Stimulation*, pp. 3419-3422.
Weiland, J. D. & Humayun, M. S., 2005. A biomimetic retinal stimulating array. IEEE Engineering in Medicine and Biology Magazine, 24(5), pp. 14-21.
Xu, S. et al., 2013. Stretchable batteries with self-similar serpentine interconnects and integrated wireless recharging systems. Nature Communications, 4, p. 1543. Available at: http://www.nature.com/doifinder/10.1038/ncomms2553.
Yamagiwa, S. et al., 2015. Chemical for low-voltage microscale electrode neurostimulation. Sensors & Actuators: B. Chemical, 206, pp. 205-211. Available at: http://dx.doi.org/10.1016/j. snb.2014.09.048.
Yang, P. K. et al., 2015. A Flexible, Stretchable and Shape-Adaptive Approach for Versatile Energy Conversion and Self-Powered Biomedical Monitoring. Advanced Materials, 27(25), pp. 3817-3824.

1. A microelectrode device with a fractal geometry shape comprising a formula of following:
ₙ=l²·5ⁿ
Pₙ=5Pₙ₋₁₁−8l(P₁=12l, n=2,3,4 . . . )
wherein (Aₙ) is the area of the microelectrode, (Pₙ) is the perimeter at iteration n with 1 as a side of the initial square.

2. The microelectrode device according to claim 1, wherein n=3.

3. The microelectrode device according to claim 1, wherein 1 is about 8 μm.

4. The microelectrode device according to claim 1 is implantable selected from the group consisting of pace makers, spinal cord, peripheral nerve, or deep brain nerve stimulators.

5. The microelectrode device according to claim 1 is platinum.

6. The microelectrode device according to claim 1 creates most current density compared to circular and serpentine type microelectrodes of similar surface area.

7. A method of making a fractal geometry shaped microelectrode, comprising the steps of:
a. Calculating a definite perimeter to area ratio pattern according to the following formula to determine the shape of the microelectrode:
ₙ=l²·5ⁿ
Pₙ=5Pₙ₋₁₁−8l(P₁=12l, n=2,3,4 . . . )
b. Spun coating a photoresist layer over a silicon nitride layer with the defined pattern in step a;
c. Depositing a Platinum film about 100nm thick on to the photoresist layer using a titanium (about 10 nm) as an adhesion layer;
d. Lifting off the photoresist layer to create the electrode arrays; and
e. Spun coating about 1.5 μm thick of polyimide over the electrode array as a passivation layer to cure.

8. The method according to claim 7, wherein the silicon nitride layer is about 500 nm.

9. The method according to claim 7, further comprising applying an adhesion promoter before spun coating polyimide.
